(19) United States
(12) Patent Application Publication
(10) Pub. No.: US 2016/0279083 A1
(43) Pub. Date: Sep. 29, 2016

(54) TOPICAL TREATMENT FOR CERVICAL INTRAEPITHELIAL NEOPLASIA

(71) Applicant: Moein Health, Inc., San Diego, CA (US)

(72) Inventors: Sudabeh Moein, San Diego, CA (US); Frederick J. Zustak, Poway, CA (US); Joshua S. Schoonover, Carlsbad, CA (US)

(73) Assignee: Moein Health, Inc., San Diego, CA (US)

(21) Appl. No.: 15/052,819

(22) Filed: Feb. 24, 2016

Related U.S. Application Data
(60) Provisional application No. 62/182,606, filed on Jun. 21, 2015.
(60) Provisional application No. 62/120,356, filed on Feb. 24, 2015.

(51) Int. Cl. A61K 31/19; A61M 31/00; A61K 9/00; A61B 1/303; A61K 31/4166; A61K 9/70

(52) U.S. Cl. CPC A61K 31/19; A61K 31/4166; A61K 9/7007; A61K 9/0036; A61B 1/303; A61M 31/00

(57) ABSTRACT

Physiologically acceptable films for use in the treatment cervical intraepithelial neoplasia are disclosed. The films include a water soluble film-forming polymer such as pullulan, and therapeutically effective amounts of chemexfoliation agents. Devices adapted to introduce these films to the cervix are disclosed, as well as various combinations of devices and films sufficient to comprise a kit to enable treatment of CIN 1, 2, and 3.

### CROSS-REFERENCE TO RELATED APPLICATIONS

This application claims the benefit of U.S. Provisional Application No. 62/182,606, filed Jun. 21, 2015, and U.S. Provisional Application No. 62/120,356, filed Feb. 24, 2015; each of which is hereby incorporated by reference.

### BACKGROUND

1. Field of the Invention

This invention relates to treatment of cervical intraepithelial neoplasia including the administration of a chemexfoliating agent; and more particularly, to compositions and therapeutic films for delivering pharmaceutically active epithelial exfoliating reagents to the cervix. In particular, the films can also be used to deliver alpha-hydroxy acids (AHA), and/or trichloroacetic acid (TCA) as a chemical exfoliant for treatment of cervical intraepithelial neoplasia (CIN) 1, 2, and 3. Further, this invention relates to devices for using the therapeutic films, and combinations of films and devices which facilitate the use of these films.

2. Description of the Related Art

Cervical cancer was the third most commonly diagnosed cancer in women in 2008, with an estimated 529,800 new cases worldwide, more than 85% of which were in developing countries.

The current method for diagnosing cervical cancer is colposcopy in which acetic acid is used as a staining agent to visualize abnormal tissue. Meanwhile, current management of patients with cervical intraepithelial neoplasia (CIN) includes the following treatments: Loop Electrical Excision Procedure (LEEP), Cold Knife Conization (CKC), cryosurgery, laser ablation, or hysterectomy. These procedures lead to partial destruction of the cervix. At the very least, the destruction will render the patient's future colposcopies unsatisfactory, because the physician will be unable to clearly visualize the squamo-columnar junction due to scarring. A more serious side effect is cervical incompetence, which poses the most impact to women of reproductive age who are still planning to have children. This destruction is of most concern for patients with recurrent disease, because additional treatments on a shorter cervix are more challenging surgically.

Despite its long history as a diagnostic procedure, colposcopy continues to have varying success. In conjunction with acetic acid, the sensitivity of colposcopy to distinguish normal from abnormal tissue is relatively high. The accuracy, however, to distinguish low-grade lesions from high-grade lesions and cancer remains low. Additionally, a substantial proportion of high-grade lesions may fail to be identified at colposcopy, resulting in no treatment and subsequent further neoplasia. In a post hoc analysis of more than 47,000 women, approximately 20% additional CIN 2 or worse and CIN 3 or worse was identified in women who did not have a visible lesion on colposcopy. Therefore the treatment of CIN I may be just as important as treating CIN II or III. However, the current above methods for removing CIN 1 abnormal tissue is traumatic, and consequently non-prescribed, largely due to the lack of a simple, non-invasive, and inexpensive procedure.

### SUMMARY OF THE INVENTION

Therapeutic compositions for treatment of cervical intraepithelial neoplasia include a chemexfoliating agent, including for example, trichloroacetic acid (TCA).

In some embodiments, physiologically acceptable films for the delivery of pharmaceutically active reagents for the treatment of cervical intraepithelial neoplasia in women (CIN 1, 2, 3) include suspensions of chemical epithelial exfoliating agents in a topical film having at least one dissolvable treatment layer. The dissolvable treatment layer immediately begins to dissolve upon contact with the moist epithelial tissue of the cervix. Suspended or dissolved agents in the dissolved film will then interact with the epithelial layer in contact with the treatment layer. In one embodiment, a therapeutic concentration of trichloroacetic acid (TCA) is dissolved or suspended in the dissolvable film, and is quickly released as the dissolvable film ablates. In another embodiment, alpha-hydroxy acids may be used as the exfoliating reagent.

Another embodiment includes a capping layer of either a non-dissolving or relatively slower dissolvable film cap, or cup, enclosing the treatment layer on the top and, optionally, on the perimeter sides of the therapeutic patch. The capping layer serves to isolate the treatment layer to just the epithelial tissue immediately underneath the treatment layer to thus prevent the TCA from migrating outside of the perimeter of the therapeutic patch which may result in possible irritation of adjacent vaginal epithelium. The capping layer's rate of dissolving is calculated to provide containment protection for at least as long as the treatment layer is still chemically active.

In one embodiment, a neutralizing reagent is provided in the capping layer to neutralize any remaining TCA or AHA after ablation of the treatment layer. When the capping is exposed to the released TCA in the ablated treatment layer, it may activate or react with an appropriate halochromic chemical compound as a pH indicator to permit visual indication and verification of final consumption of the exfoliating reagent and the end of the physiological activity. In order to ensure total consumption of the TCA or AHA, a neutralizing agent may be incorporated in the capping layer as well, or in an intermediate neutralizing layer. The neutralizing layer is disposed intermediate of the treatment layer and the capping layer. The neutralizing agent is released during the ablation of the neutralizing layer, and after the ablation of the treatment layer, to ensure neutralization of the TCA or AHA, the neutralizing agent being provided in a concentration sufficient to neutralize an anticipated amount of TCA or AHA residual.

In another embodiment, the capping layer or the neutralizing layer may also contain allantoin to provide a moisturizing and keratolytic effect, increasing the water content of the extracellular matrix and enhancing the desquamation of upper layers of dead skin cells resulting from the physiological activity of the TCA or AHA. Also, the allantoin will increase the smoothness of the replacement epithelial skin layer, promote cell proliferation and wound healing; and provide a soothing, anti-irritant, and skin protectant effect by forming complexes with any remaining reagents. The allantoin is released upon ablation of the neutralizing layer, or the capping layer. In an embodiment where the capping layer is not-dissolvable, the allantoin may be made available by leaching the allantoin from a porous capping layer matrix.

In yet another embodiment of the therapeutic patch of this invention, the capping layer may be a non-dissolving layer that includes an inside surface that when adjacent to the dead epithelial cells, will have a mechanical or chemical attachment system for attaching to the dead cells and allowing easy removal of the dead cells upon extraction of the undissolved capping layer. This layer may also include a pH indicator for visually confirming cessation of reagent activity.

In another embodiment the dispersion of the suspended reagent may be uniform throughout the reagent layer thus providing an even introduction of the TCA or AHA over time and surface area. The reagent may also be dispersed, or suspended, to provide a vertical concentration gradient to thus provide time dependent reagent delivery rate to the treatment area. Alternately, or additionally, the reagent may be dispersed, or suspended to provide a radial concentration gradient to focus treatment on a particular area (e.g., the cervix opening) while providing lesser or no treatment in other areas covered by the film.

In another related aspect of this invention, an insertion device for attaching the film of this invention includes a concave cup to conform to the generally convex, or toroidal shape of the cervix, the cup having at least an opening for vacuum clamping a therapeutic film of this invention, and a vacuum relief for releasing the film once the film is attached to the cervix. In an alternate embodiment of the insertion device, the therapeutic patch may be provided releasable pre-attached to the concave surface of the insertion tool, which can release the patch once the patch is introduced to the cervix, or the insertion tool left in place while the treatment layer is ablated, and then removed when the treatment layer has sufficiently completed its ablation.

In yet another related aspect of this invention, an assemblage of therapeutic patches, insertion devices, various swabs, saline solution, and, other devices such as speculums are included in a convenient kit in sufficient quantities to provide a complete course of treatment for a single patient therapy session. Variants of the kit will include therapeutic patches having various physical criteria suitable for the condition to be treated. Other variants of the kit include an auxiliary kit adaptable to be included in addition to the above described kit. The auxiliary kit will include solutions, such as Monsel paste and Lugols® solution, and devices, such as a speculum light that can be shared across multiple patient visits.

### BRIEF DESCRIPTION OF THE DRAWINGS

To accomplish the foregoing and related ends, certain illustrative embodiments of the invention are described herein in connection with the following description and the annexed drawings. These embodiments are indicative, however, of but a few of the various ways in which the principles of the invention may be employed and the present invention is intended to include all such aspects and their equivalents. Other advantages, embodiments and novel features of the invention may become apparent from the following description of the invention when considered in conjunction with the drawings. The following description, given by way of example, but not intended to limit the invention solely to the specific embodiments described, may best be understood in conjunction with the accompanying drawings, in which:

FIG. 1 is a perspective view of one embodiment of the therapeutic patch of this invention;

FIG. 2 is a cross-section view of the treatment layer of the therapeutic patch having a uniform concentration gradient of reagent;

FIG. 3 is a cross-section view of the treatment layer of the therapeutic patch having a radial concentration gradient of reagent;

FIG. 4 is a cross-section view of the treatment layer of the therapeutic patch having a vertical concentration gradient of reagent;

FIG. 5A is a cross-section view of the treatment layer and capping layer of a therapeutic patch;

FIG. 5B is a cross-section view of the treatment layer and capping layer of a therapeutic patch having no perimeter sides;

FIG. 6 is a cross section view of the therapeutic patch with treatment layer, a neutralizing layer, and a capping layer;

FIG. 7 is a cross section view of the therapeutic patch with treatment layer, a neutralizing layer, a capping layer, and a clean-up layer;

FIG. 8 is a cross section view of a therapeutic patch insertion tool;

FIG. 9 is a perspective view of a therapeutic patch insertion tool;

FIG. 10 is a cross section view of a therapeutic patch insertion tool;

FIG. 11 is a cross section view of a therapeutic patch insertion tool head;

FIG. 12 is a cross section view of a modified therapeutic patch insertion tool head; and

FIG. 13A is a plan view of a therapeutic patch kit tray;

FIGS. 13B is a perspective view of the underside of a therapeutic patch kit tray;

FIG. 14 is perspective exploded view of the therapeutic patch kit tray and cover;

FIG. 15 is perspective view of the therapeutic patch kit tray cover; and

FIG. 16 is a perspective exploded view of the therapeutic patch kit tray, cover, and auxiliary nesting tray.

### DETAILED DESCRIPTION OF THE PREFERRED EMBODIMENTS

The following embodiments are described in sufficient detail to enable those skilled in the art to make and use the invention. It is to be understood that other embodiments would be evident based on the present disclosure, and that system, process, mechanical, or chemical changes may be made without departing from the scope of the present invention.

In the following description, numerous specific details are given to provide a thorough understanding of the invention. However, it will be apparent that the invention may be practiced without these specific details. In order to avoid obscuring the present invention, some well-known compositions, system configurations, and process steps are not disclosed in detail.

The drawings showing embodiments of the system are semi-diagrammatic and not to scale and, particularly, some of the dimensions are for the clarity of presentation and are shown exaggerated in the drawing figures.

Where multiple embodiments are disclosed and described having some features in common, for clarity and ease of illustration, description, and comprehension thereof, similar and like features one to another will ordinarily be described with similar reference numerals. The embodiments have been numbered first embodiment, second embodiment, etc. as a matter of descriptive convenience and are not intended to have any other significance or provide limitations for the present invention.

The term “therapeutic patch” or “therapeutic film” referred to herein are synonymous and can include single layer, or multi-layer patches, or equivalently “thin films” for application to a dermal surface, The therapeutic patch may be the patch itself, or include an insertion tool to which the patch is affixed, or a combination thereof. For example, the therapeutic patch can be a single or multi-layer composition, or a single or multi-layer composition removably bonded to an insertion tool for facilitating the application of the composition to an affected dermal area. The term “film” as referred to herein refers to that portion of the therapeutic patch in which current context of the description applies.

The term “treatment layer” referred to herein means either a single layer of a therapeutic patch that contains the active reagent for performing the treatment once it has made contact with the affected dermal layer. In one embodiment the treatment layer includes an exfoliating reagent that is either dissolved in a supporting solid substrate, or a dispersed solid or microencapsulated reagent in a supporting substrate.

The term “substrate” referred to herein means the composition in which the active reagent is either dissolved or dispersed as a solid suspension. If a solid suspension then the term “matrix” may be used to describe the composition surrounding and supporting the solid active reagent. The term “dissolvable film” is contextually related to the term substrate or matrix, but more specifically includes the characteristic of being a thin film configured to be a patch that may be applied to a predetermined area of dermis, and being dissolvable in the presence of moisture.

The term “reagent” is synonymous with the term “active reagent” and refers to the non-inert component of a layer in the therapeutic patch. For example, in the treatment layer, an exfoliating reagent such as trichloroacetic acid (TCA) may be dissolved as a liquid in a substrate of a water soluble gel, or the TCA may be dispersed as a solid suspension in a water soluble matrix.

The term “capping layer” referred to herein means an outer layer of the therapeutic patch that is the most distal layer from the affected dermis.

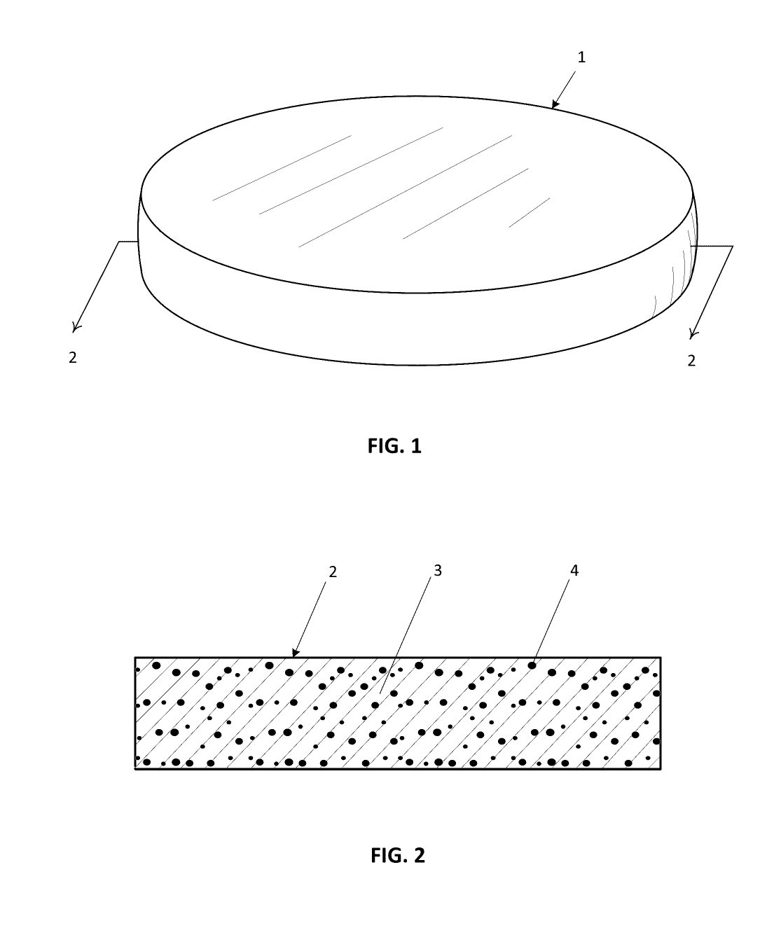

Referring initially to the non-limiting example embodiment shown in FIG. 1, a therapeutic patch 1 is sized to cover the cervix, and deliver a predetermined dose of a chemexfoliating reagent to the epithelial dermis of the cervix. FIG. 2 is a cross section view of a therapeutic patch of this and includes a treatment layer 2 of a physiologically compatible dissolvable matrix 3 having suspended therein a therapeutic amount of the active reagent 4. The dissolvable matrix may be selected from a variety of dissolvable film materials having different adhesion characteristics to moist epithelial tissue, dissolving rates, and agent loading capacities. A popular physiologically compatible film is pullulan. Pullulan is a polysaccharide polymer produced from starch by the fungus Aureobasidium pullulans. Pullulan is mainly used by the cell to resist against desiccation and predation, the presence of this polysaccharide also facilitate diffusion of molecules both into and out of the cell. The chief commercial use of pullulan is in the manufacture of edible films that are used in various breath freshener or oral hygiene products such as Listerine Cool Mint™ of Johnson and Johnson® (USA) and Meltz Super Thin Mints™ of Avery Bio-Tech Private® Ltd. (India). As a food additive, it is known by the E number E1204. Pullulan would be a suitable material for the dissolvable film of the treatment layer. Other dissolvable, physiologically compatible film materials may be obtained from Adhesives Research, Inc.™, a leading independent developer and manufacturer of pressure-sensitive adhesives (PSAs), polymers, tapes, films, coatings, laminates, release liners and drug delivery systems. See http://www.adhesivesresearch.com/healthcare/. The physiologically compatible dissolvable film chosen should have excellent tack upon contact with the epithelium of the cervix, and dissolve very quickly to deliver the chemexfoliating agent.

Other suitable films may comprise water soluble polymers, or polymers soluble both in water and organic solvents, such as hydroxypropyl cellulose (HPC) and the like. The film is sized to cover the outer dermal surface of the cervix, but not to extend onto the vaginal epithelium.

The chemexfoliating reagent 4 of the treatment layer 2 of the therapeutic film 1 is selected from chloroacetic acids, including preferably trichloroacetic acid (TCA), and alpha hydroxy acids (AHA) (e.g., glycolic acid, lactic acid, malic acid, citric acid, and tartaric acid).

Effective concentrations of TCA in the treatment layer should be equivalent to between 50% and 100% w/v solutions, and preferably between 60% and 90% w/v. For a 25 mm treatment film that is one mm thick, the amount of TCA loading that is equivalent to a ½ milliliter dose of 85% TCA w/v is approximately 0.8 grams of TCA.

If AHA is used in the treatment layer, the amount of AHA dispersed in the dissolvable film dispensed by the treatment layer should be the equivalent of a volumetric dose of approximately ½ milliliter of 60%-90% AHA w/v solution.

The dissolving substrate should be chemically compatible with the TCA (i.e., non-reactive with the TCA), or whichever chemexfoliating agent is chosen, particularly if the exfoliating reagent is dissolved in the substrate material. As shown in FIG. 2 the TCA 4, is compatible may be a solid suspension of the anhydrous crystalline powder, a solid suspension of encapsulated TC crystalline powder, or a suspension of microencapsulated liquid TCA solution. As the TCA is very hydroscopic, encapsulating the solid TCA in a material that does not transport water vapor may be desirable. Alternately, manufacture and packaging of the therapeutic film should preclude absorption of ambient water vapor and subsequent early degradation of the therapeutic film. Such packaging may include individual water vapor impermeable packets intended to have a long shelf life. Such packets may be hermetically sealed foil-lined plastic packets.

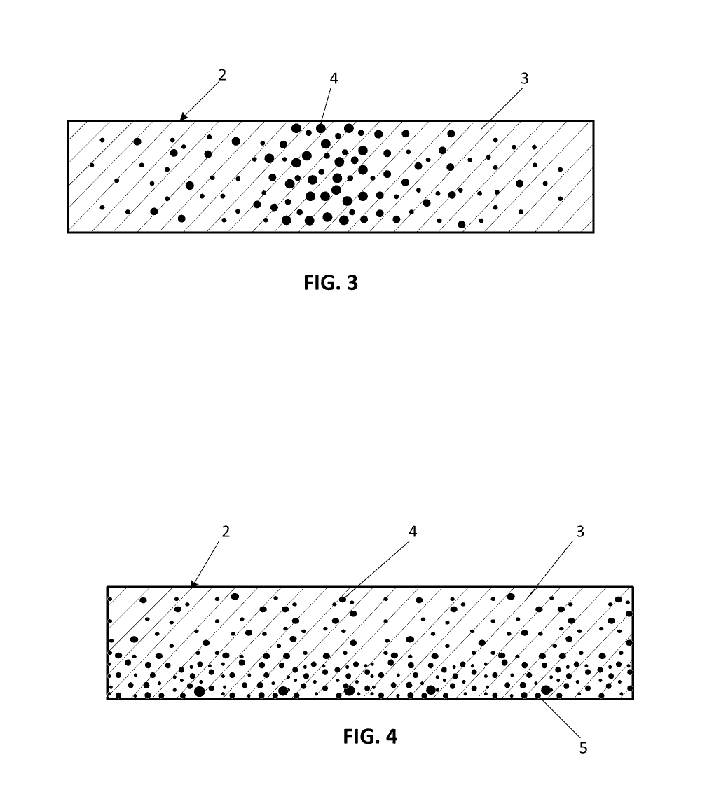

The TCA may be dispersed evenly throughout the dissolvable film as shown in FIG. 2, or it may be distributed so as to create a concentration gradient of the TCA in the film. FIG. 3 shows a cross section of a treatment layer 2 where the TCA 4 is distributed in the substrate 4 to provide a solid suspension, or solution, that has a radial gradient. This may be useful if more of the TCA reagent is needed in a localized area of the film; e.g., at the cervical opening. To achieve this end, multiple layers of the film can be independently manufactured, with each of the layers having TCA applied with a distinct radial gradient. For example, in a first layer of the film the TCA may extend radially out to one/half inch from a center of the film, and in a second layer of the film the TCA may extend radially out to about one inch from the center. With multiple layers, the concentration of TCA can be distributed in a radial gradient. In another embodiment, the TCA can be aspirated about the film using a jet-aspiration device, wherein the TCA is delivered in higher concentration near the center of the film and gradually tapered as the TCA is delivered radially outward from the center.

In one embodiment, a water-dissolvable therapeutic film is prepared in accordance with the above descriptions and that which is known in the art. Crystalline TCA powder having a particle size of one hundred microns or less, and preferably fifty microns or less, is applied to at least one surface of the film, the surface later to be the dermis side of the therapeutic patch. The film and TCA powder may be further coated with a dissolvable layer, such as with a dissolvable polymer coating. In this regard, a dissolvable film is provided with at least one surface of the film being coated with TCA powder and an optional water-dissolvable coating. FIG. 4 likewise shows a treatment layer 2 wherein the TCA reagent 4 is distributed in the dissolvable substrate 3 to provide a vertical concentration gradient. This may be useful if a rapid initial reaction is desired, or if a higher concentration is needed later during the treatment as the dead skin cell layer becomes deeper.

In another embodiment, a water-dissolvable thin film is prepared in accordance with the above and that which is known in the art. Crystalline TCA powder is introduced or embedded within the thin film prior to drying. For example, TCA powder can be encapsulated in a carbohydrate glass, such as glucose or other sugar-glass matrix, and the coated TCA particles can be introduced into the polymer film prior to drying. Some sugars are known to be insoluble in acetone and certain other organic solvents. Upon encapsulating the TCA particles in carbohydrate-glass, the encapsulated TCA particles can then be introduced into an organic solvent such as acetone without dissolving the TCA particles. Thus, a polymer such as hydroxypropyl cellulose (HPC), which is bi-soluble in both organic solvents (ex: acetone) and water, can be used to manufacture the films. In sum, HPC is dissolved in acetone forming a polymer solution. TCA powder is encapsulated in a carbohydrate-glass, and the encapsulated TCA powder is introduced into the polymer solution and mixed. Note that the encapsulated TCA will not dissolve in the organic solvent because it is coated with a protective encapsulating layer of sugar-glass (which is not soluble in acetone). The polymer solution and suspended TCA powder is then cast and dried to form the film. The resulting film will include the carbohydrate-glass coated TCA powder suspended in the polymer substrate. Now, with exposure to a moist environment, such as the cervix of a patient, the film which is also water-soluble will begin to ablate, thereby exposing the carbohydrate-glass coated TCA powder to the cervix, the carbohydrate glass is also soluble in water and thus begins the erosion of the carbohydrate-glass encapsulating layer thereby exposing the TCA powder, which in the moist environment will dissolve into a liquid-phase acid for treating a surface layer of epithelial cells about the cervix.

In yet another embodiment, an amount of liquid TCA is introduced into an aqueous solution containing a water-soluble polymer prior to drying. Films are cast or otherwise manufactured from the polymer solution.

In addition to HPC, certain forms of cellulose acetate may provide a suitable bi-soluble polymer for forming water-dissolvable films.

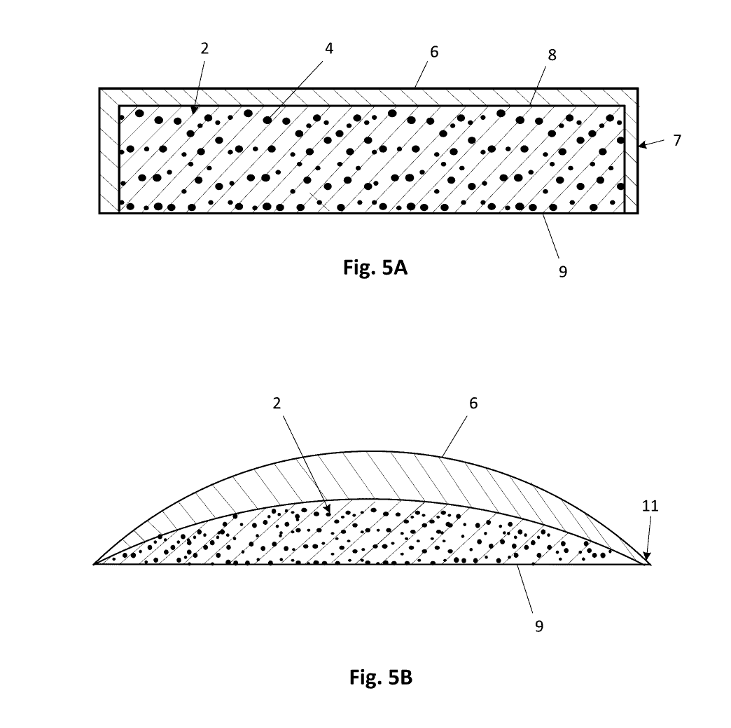

FIG. 5A shows a therapeutic patch having a treatment layer 2, and a capping layer 6. The capping layer 6 may be another dissolvable film having much slower dissolving rate as compared to the treatment later. The purpose of the capping layer is to seal in the treatment layer and isolate the treatment layer away from adjacent sensitive tissue, such as the vaginal epithelium. Additional functionality may be imparted to the capping layer by including pH indicator to provide visual confirmation that the treatment layer has been activated and the TCA is going into solution. It can also provide a visual indication of when the TCA has been exhausted.

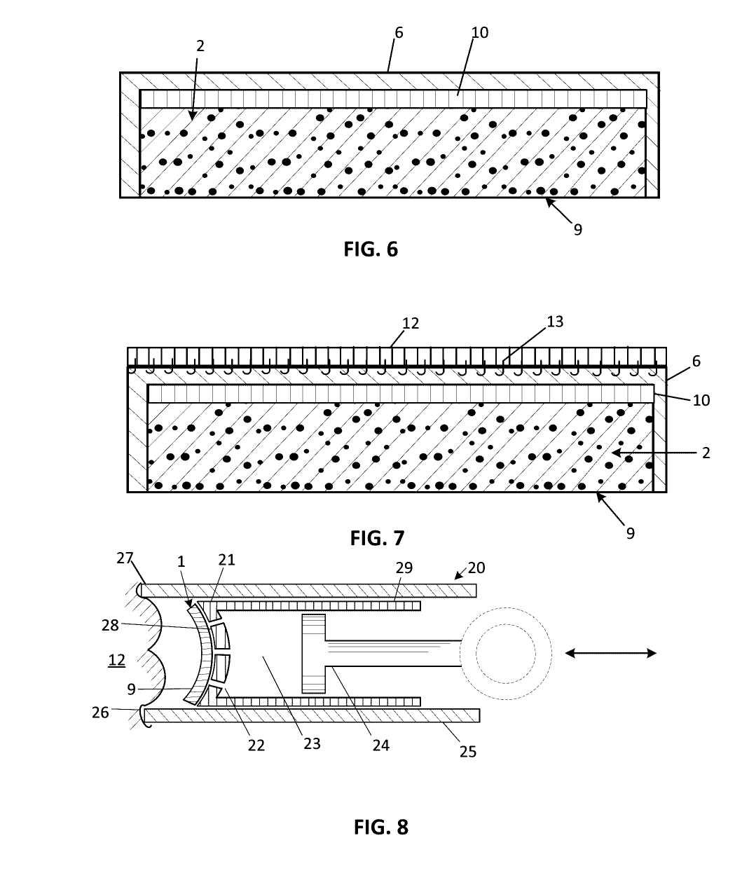

The therapeutic patch of FIG. 5A may also include a neutralizing reagent in the capping layer such that any residual TCA 4 from the ablated treatment layer 2 will be pH neutralized to thereby truncate the time needed for a therapy session. If the neutralizing agent is released after 5 minutes, that would have given the TCA plenty of time react with the epithelial cells of interest. FIG. 6 is a therapeutic film that includes a separate neutralizing layer 10 disposed between the treatment layer 2 and the capping layer 6. This may be useful to ensure any residual TCA or other chemexfoliant is neutralized prior to confirm such by a pH indicator in the capping layer, or before dispersing allantoin from the capping layer.

The therapeutic patch of FIG. 5A may also contain a suspension of, or solubilized Allantoin in the capping layer 6. Alternately, the Allantoin may be dissolved or dispersed in the neutralizing layer 10 of FIG. 6. Allantoin has a moisturizing and keratolytic effect, increasing the water content of the extracellular matrix and enhancing the desquamation of upper layers of dead skin cells, increasing the smoothness of the skin; promoting cell proliferation and wound healing; and a soothing, anti-irritant, and skin protectant effect by forming complexes with irritant and sensitizing agents. Inclusion of Allantoin in the capping layer 6, or the neutralizing layer 10 may expedite recovery of the new skin cells, and shorten the time between TCA treatments. 5% Allantoin ameliorates the wound healing process, by modulating the inflammatory response, and promotes fibroblast proliferation and synthesis of the extracellular matrix. This will serve to shorten the time interval between treatments, and to promote more complete recovery. Concentrations of allantoin may range from 1% w/v to 10% w/v.

The capping layer 6 may be extended to not only cover the distal surface 8 of the treatment layer 2, but the perimeter walls 7 of the treatment layer as well as shown in FIG. 4. A fast tack characteristic of the dermis side 9 of the treatment layer to the moist dermis of the cervix will result in complete isolation of the treatment layer 2 within the capping layer thus preventing seepage of the chemexfoliating agent to adjacent healthy tissue and the concomitant irritation. The capping layer may include one or more of a pH indicator, a neutralizer, and allantoin to perform the ancillary functions as described above.

Additionally, one or more biologically acceptable tacking agents can be embodied in the capping layer to form a tacky paste or similar layer when the treatment layer is completely dissolved. Any known tacking agent that is suitable for topical biological application may be used. In some embodiments, it may be desirable to provide carbohydrate-glass encapsulated hydrogel microspheres as an exemplary tacking agent. Similar to the carbohydrate-glass encapsulated TCA, the carbohydrate-glass encapsulating layer will erode in the moist environment of the cervix, exposing the hydrogel microspheres which then absorb moisture and form a tacky layer covering the application site.

Thus, in a multi-layer film, a first layer may include a treatment layer comprising TCA or AHA; whereas a second layer may include a tacking agent such as carbohydrate-glass encapsulated hydrogel microspheres. In this embodiment, the second layer forms the “capping layer” of the film. Because it is important to apply the first layer to the surface of the target delivery site, the first and/or second layers can be individually marked to identify the surface which should be applied. The marking may consist of any visual representation, preferably a color or marking.

In another embodiment, and as shown in FIG. 5B, the capping layer is configured to substantially cover the treatment layer, wherein the capping layer does not comprise perimeter walls. In this embodiment, the capping layer may comprise a periphery 11 that extends equally or beyond the perimeter periphery of the treatment layer 2; an overlapping capping layer 6, and a periphery 11 that is equal to or greater than a periphery of the treatment layer. In some embodiments, and as shown in FIG. 5B, the capping layer may form a dome shape layer with the treatment layer disposed in a volume thereof.

FIG. 7 is an embodiment including a non-dissolving “clean-up” layer 12. Once all of the intermediate layers have dissolved, micro-hooks 13, or a chemical adhesive attaches to the dead skin, and removal of the cleaning layer will also remove the dead skin layer thus facilitating prepping for follow up treatment. Micro-hooks be produced by casting one or more layers of the film.

As the treatment layer surface is very hygroscopic and self-attaches itself and start dissolving upon contacting any moist epithelial surface, including the vaginal walls, it is desirable to isolate the treatment layer's exposed surface from intermediate epithelial surfaces upon insertion of the therapeutic film through the vaginal canal. The insertion tool 20 of FIG. 8 includes a concave cup 21, of a radius approximately similar to the cervix radius. The cup is pierced and includes air ways 22 extending from the distal surface 28 or patch side of the cup and to a suction chamber 23. A plunger 24, or a bulb (not shown) cause a partial vacuum to form in the suction chamber when a therapeutic patch 1 is placed on the concave surface, and the plunger 24 is manipulated to pull a vacuum in the suction chamber via the airways 22. The partial vacuum in the suction chamber will thus affix the therapeutic patch 1 to the concave surface 28, and permit the housing 25 to be inserted into the vaginal canal without exposing the therapeutic patch to the vaginal epithelium. The insertion tool 20 is position such the distal end 27 of housing 25 is placed against the circular crease 26 defined by intersection of the cervical epithelium, and the vaginal epithelium. This permits precise positioning of the therapeutic patch against the cervical epithelium, and avoiding contact with the vaginal epithelium. The plunger 24 when depressed, will release the vacuum, thus releasing the therapeutic patch onto the cervix. The insertion tool is withdrawn leaving the therapeutic film in place. In another embodiment, the walls 20 of the insertion tool tube may be replaced by using a speculum and guiding just the suction chamber having its own walls 29 through the speculum.

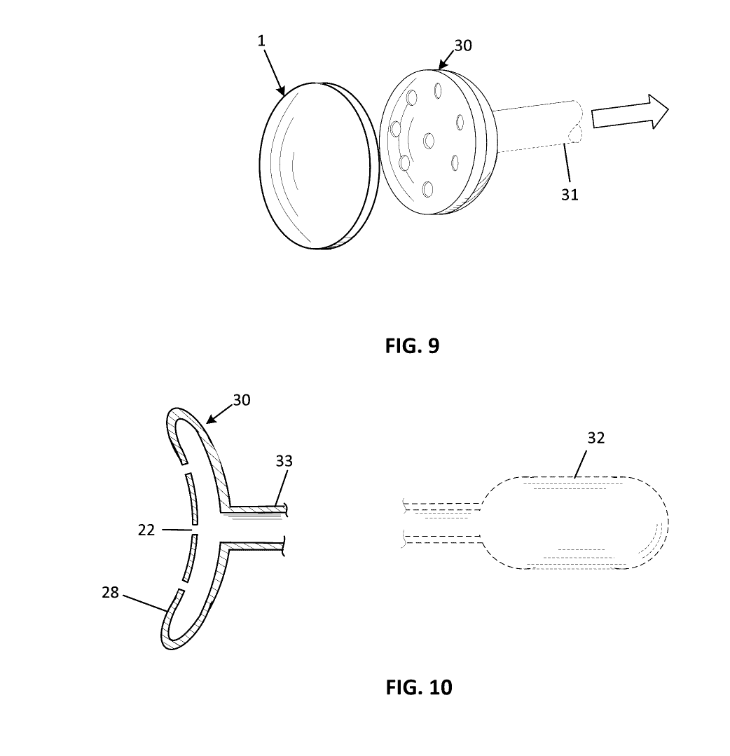

The insertion device shown in FIG. 9 includes a hollow front cup portion, having a foraminous distal surface 28. The insertion device can be pneumatically connected to a disposable pipette (not shown) having a bulb on its proximate end for drawing in fluids, or other suitable device capable of drawing a slight vacuum. When a therapeutic patch is placed over the distal face 28 of the cup, operation of the pipette bulb will create a slight vacuum in the hollow cup and vacuum clamp the therapeutic patch to the distal surface 28. The insertion tool with the therapeutic patch affixed can now be guided down the speculum, and the therapeutic patch placed directly over and against the cervical epithelium. The vacuum in the hollow cup 30 is released thus releasing the therapeutic patch onto the cervix. The front cup portion may be curved to conform to a cervix surface, or concave, or may comprise a planar surface.

A cross section view of the insertion device of FIG. 9 is shown FIG. 10. A bulb 32 capable of drawing a slight vacuum is shown in phantom. In operation a therapeutic patch is placed over the distal surface 28 of the cup 30 such that it is removably affixed to the distal surface via the airways 22 when a slight vacuum is drawn. A connection port 33 permits pneumatic connection to a vacuum source such as the bulb 32.

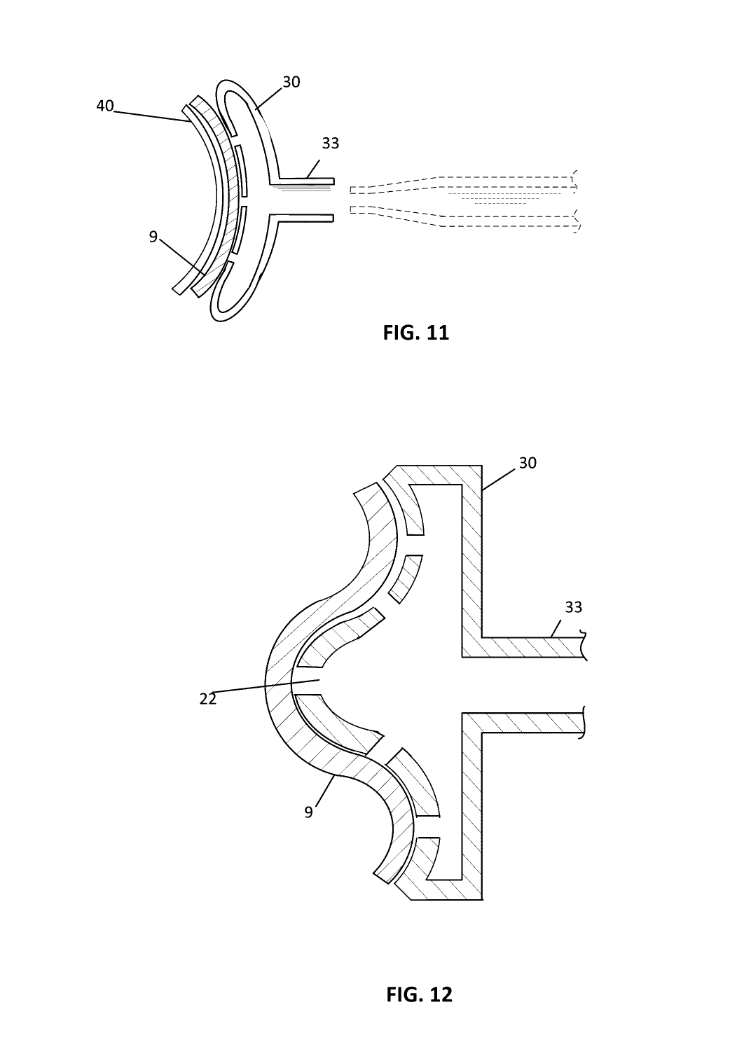

In another embodiment, the therapeutic film may come pre-attached to an insertion device as shown in FIG. 11 where the cup 30 having a foraminous distal surface is packaged with a therapeutic patch pre-attached thereto with the cervix side of the treatment layer 9 position for deployment on the cervical epithelium. The pre-attached patch may have a disposable peelable protective layer 40 that can be removed and discarded prior to use. In operation, protective layer 40 is peeled off the pre-attached therapeutic patch and discarded. A port 33 permits pneumatic connection to a pipette having a bulb that will create a positive pressure inside the cup, to thus release the therapeutic patch once it is positioned over and against the cervix.

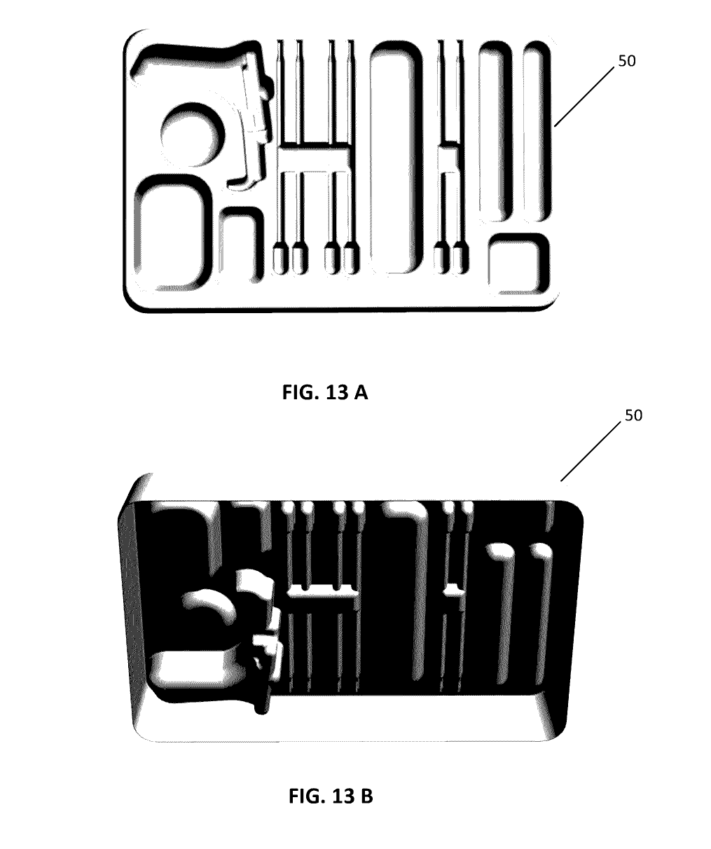

In another embodiment, the insertion tool may include a hollow cup portion 30 as shown in FIG. 12 where the distal surface 28 of the cup is configured to place the therapeutic patch at least part way into the cervical opening, or external os. As in the embodiment shown in FIG. 11, the therapeutic patch 1 may be pre-attached and provided as patch and cup assembly. As in the embodiment, the pre-attached therapeutic patch will have a protective peelable layer (not shown) covering the cervix side of the treatment layer 9. In an alternate embodiment a therapeutic patch having the TCA dispersed in a radial concentration gradient is pre-attached to the insertion tool of the embodiment of FIG. 12 to further concentrate treatment in the external os region. Operation of this embodiment is similar to the operation described above for the embodiment FIG. 11.

FIG. 13A is a plan view of a basic kit tray for accommodating the equipment and reagents necessary for performing a therapeutic treatment should cervical neoplasia be detected. The tray 50 is ideally vacuum formed and includes at least compartments for a collection components necessary for an individualized treatment kit for minor cervical neoplasia, including therapeutic patches, insertion tool, speculum, saline solution, silver nitrate hemostat sticks, and swabs, pipettes, Lugol's solution, and other related items necessary for a single course of treatment. FIG. 13 B is a perspective view of the rear of the kit tray. By vacuum forming the tray 50, manufacturing costs are minimized, and the tray can be easily modified to accommodate changes in the kit.

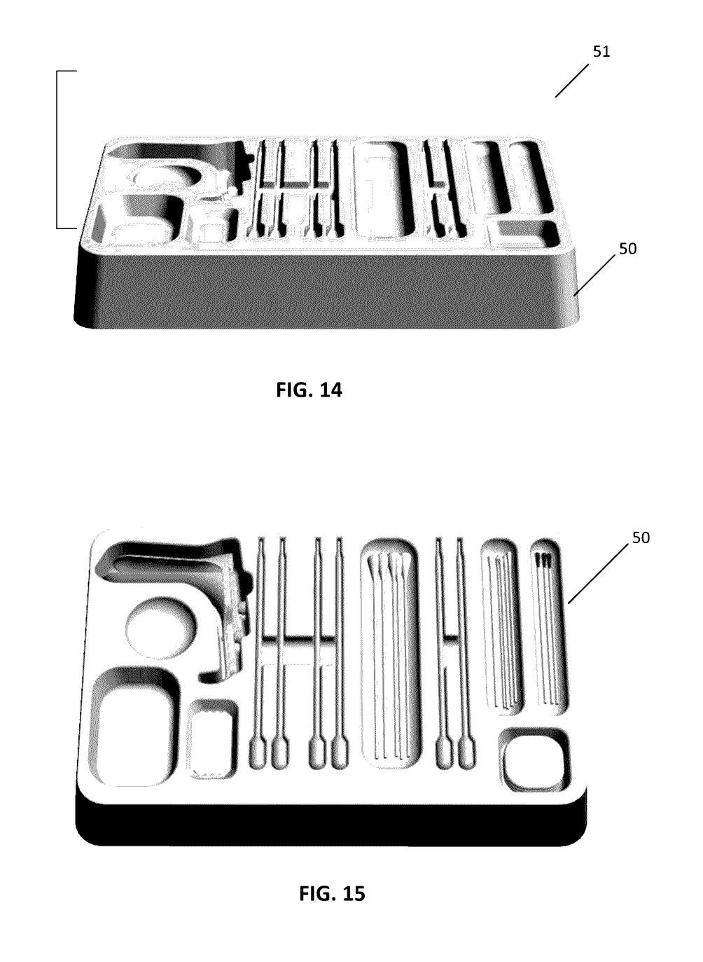

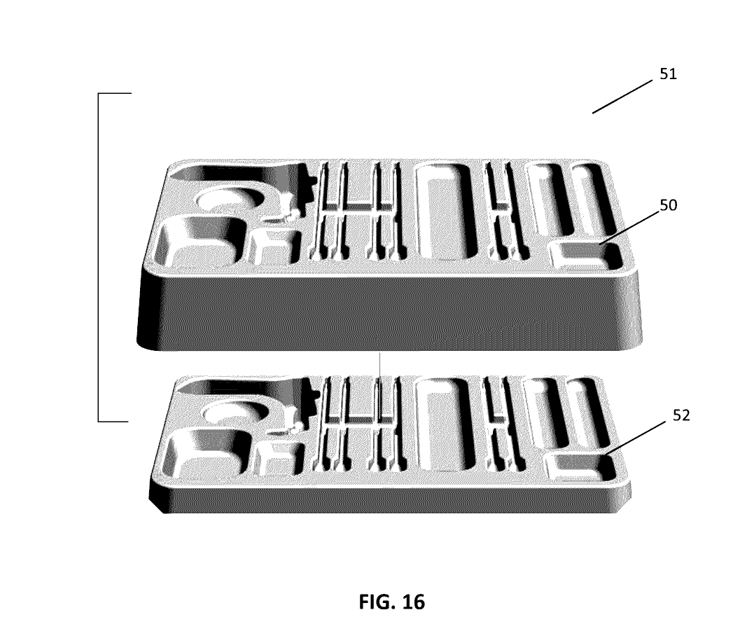

FIGS. 14 and 15 show the therapeutic basic kit of this invention including a sealed peelable cover 51 for sealing the kit tray 50 during transport and storage. FIG. 15 shows the tray populated with items conveniently provided and arranged to facilitate a therapeutic treatment session of a mild cervical neoplasia. FIG. 16 is an exploded perspective view of the basic kit tray for accommodating the components and solutions necessary to conduct routine therapeutic treatment of minor neoplasia. On occasion, however, should a biopsy of suspected tissue result in bleeding that cannot be stopped with the silver nitrate hemostat sticks (caustic pencils), then additional materials may be needed. An auxiliary tray 52 containing compartments for accommodating additional components typically desired in the event of a contingency is provided in a more advanced kit. This tray is designed to nest into the basic kit tray, thus not taking up any additional volume over the basic kit. The auxiliary kit will provide a compartment for Monsel paste, or Monsel ampoules, such as Stip Tik Swabpoules™ by Health Science Laboratories, Phoenix, Ariz. In addition a speculum light may be included that would provide illumination for the speculum provided in the basic kit.

In other embodiments, a therapeutic composition comprising a chemexfoliating agent is applied to a cervix of the patient. A method for administering the chemexfoliating agent may comprise, in any order: (i) providing a therapeutic composition comprising a chemexfoliating agent; (ii) first applying an initial amount of the therapeutic composition to at least a portion of a cervical epithelium of a patient; (iii) at least forty eight hours after said first applying: second applying a subsequent amount of the therapeutic composition to the at least a portion of the cervical epithelium of the patient. The method may further comprise: placing the patient in a dorsolithotomy position; introducing a speculum into a vagina of the patient to expose a cervix of the patient, the cervix including the cervical epithelium; clearing discharge or secretion using a swab; visualizing the cervix for gross abnormalities; removing the speculum from the vagina of the patient; or a combination thereof.

The method of claim 17, wherein said chemexfoliating agent comprises trichloroacetic acid (TCA).

The method of claim 19, wherein said chemexfoliating agent comprises TCA in a composition of between 45% and 85% weight to volume in aqueous solution.

Other variations of the instant disclosed features and embodiments, particularly when combined with other features and methods known in the art, are deemed to be included within the spirit and scope of this disclosure.

What is claimed is:

1. A therapeutic patch for treating cervical neoplasia, comprising:
a treatment layer, the treatment layer comprising a water dissolvable substrate and a chemexfoliating reagent.

2. The therapeutic patch of claim 1, wherein the chemexfoliating reagent is selected from the group consisting of: trichloroacetic acid (TCA), glycolic acid, lactic acid, malic acid, citric acid, and tartaric acid.

3. The therapeutic patch of claim 1 where in the chemexfoliating reagent is trichloroacetic acid (TCA).

4. The therapeutic patch of claim 1 further comprising a capping layer, said capping layer configured to contain an ablated treatment layer to protect adjacent vaginal epithelium from the chemexfoliating reagent.

5. The therapeutic patch of claim 2 wherein said capping layer further comprises a pH indicating reagent to give visual indication of a change of pH in the ablated treatment layer.

6. The therapeutic patch of claim 2 wherein said capping layer further comprises allantoin.

7. The therapeutic patch of claim 2 further comprising a neutralizing layer for neutralizing the chemexfoliating reagent after ablation of the treatment layer is completed.

8. The therapeutic patch of claim 7 further comprising allantoin in the neutralizing layer.

9. (canceled)

10. (canceled)

11. (canceled)

12. (canceled)

13. A therapeutic kit for treating cervical neoplasia, comprising at least one therapeutic patch, at least one insertion tool, and a speculum.

14. The therapeutic kit of claim 13, further comprising a component tray, and a peelable protective cover, the component tray configured with one or more compartments for accommodating single use therapy components, the single use therapy components comprising at least one therapeutic patch, at least one insertion tool, and a speculum.

15. The therapeutic kit of claim 13, further comprising one or more: transfer pipettes, saline solution, swabs, Monsels paste, lubricating jelly, speculum light, Monsel ampoules, gauze, or a combination thereof.

16. The therapeutic kit of claim 14, further comprising a nesting auxiliary tray having compartments for solutions and devices that may be used multiple times.

17. A method for treatment of cervical intraepithelial neoplasia, comprising in any order:
providing a therapeutic composition comprising a chemexfoliating agent;
first applying an initial amount of the therapeutic composition to at least a portion of a cervical epithelium of a patient; and
at least forty eight hours after said first applying:
second applying a subsequent amount of the therapeutic composition to the at least a portion of the cervical epithelium of the patient.

18. The method of claim 17, further comprising one or more of:
placing the patient in a dorsolithotomy position;
introducing a speculum into a vagina of the patient to expose a cervix of the patient, the cervix including the cervical epithelium;
clearing discharge or secretion using a swab;
visualizing the cervix for gross abnormalities; and
removing the speculum from the vagina of the patient.

19. The method of claim 17, wherein said chemexfoliating agent comprises trichloroacetic acid (TCA).

20. The method of claim 19, wherein said chemexfoliating agent comprises TCA in a composition of between 45% and 85% weight to volume in aqueous solution.
